(19) United States
(12) Patent Application Publication
(10) Pub. No.: US 2018/0214585 A1
(43) Pub. Date: Aug. 2, 2018

(54) DISINFECTION DEVICES AND SYSTEMS

(71) Applicant: Patrick J. Piper, Kansas City, MO (US)

(72) Inventor: Patrick J. Piper, Kansas City, MO (US)

(21) Appl. No.: 15/886,808

(22) Filed: Feb. 1, 2018

Related U.S. Application Data
(60) Provisional application No. 62/453,411, filed on Feb. 1, 2017.

(51) Int. Cl. A61L 2/10; C02F 1/32

(52) U.S. Cl. CPC A61L 2/10; A61L 2202/11; C02F 1/325

(57) ABSTRACT

The invention provides a variety of devices, systems, and methods that emit a specific range of ultraviolet radiation towards one or more surfaces that it is desirable to disinfect from microorganisms or allergens.

### BACKGROUND OF THE INVENTION

A. Field of the Invention

The present invention relates to improved disinfection devices and their associated uses and methods that use of a narrow spectrum of ultraviolet light to eliminate biological contaminants.

B. Description of the Related Art

Implementing, operating, and maintaining a healthy and safe environment is of paramount importance in any facility, as well as, areas that are likely to be sources of various pathogens spread through contact. Current disinfection solutions are costly, require time-consuming processes and have limited efficacy. The level and type of infectious agents and other contaminants are also factors contributing to costs and challenges in maintaining safe and healthy environments.

Ultraviolet (UV) radiation or light has been known to be germicidal for over 100 years and has been broadly used for disinfection since the mid-20th century. It is used for drinking and wastewater treatment, air disinfection, the treatment of fruit and vegetable juices, as well as, a myriad of home devices for disinfecting everything from toothbrushes to phones and other electronics. While UV radiation can be a very efficient bactericidal agent, it is also known to have mutagenic and carcinogenic properties that prevent widespread use of the technique.

Typically, when UV irradiation has been used to disinfect food, air, or water, short wavelengths of UV light, typically in the UVC (wavelengths 240 to 280 nm) range, have been used. Such UV irradiation is typically produced with low-pressure mercury lamps, which can produce a range of UV wavelengths, ranging from UVA (wavelengths 315 to 400 nm) to UVB (wavelengths 280 to 315 nm) or to UVC. Commonly, mercury-vapor lamps emit UV radiation at around 254 nm, which may be harmful to humans and other life forms. These mercury-vapor lamps are typically shielded or in environments where exposure is limited to reduce or prevent the health hazards associated with their use. Thus, depending upon the environment, the utility of such lamps is limited or excluded.

Ultraviolet germicidal irradiation with light emitting diodes (LEDs), plasma and excimer lamps is now being researched and developed for commercialization. An excimer lamp can produce a wide high-intensity narrow range of wavelength UV radiation. These lamps can be small, relatively inexpensive, long-lived (e.g., 1,000 or more hours) and high powered so that disinfection can take place rapidly. Reducing or eliminating the presence of various pathogens and other contaminants in air, water, and surfaces would likely yield safer, healthier environments. Novel, green devices are needed to insure the safety of users and promote more efficient operational procedures in facilities.

### SUMMARY OF THE INVENTION

The present invention provides improved disinfection devices, as well as their associated methods, that use a narrow spectrum of far ultraviolet light to eliminate biological contaminants such that safety deficiencies in the art are overcome. Advantageously, devices of the present invention do not require shielding to protect users from harmful ultraviolet (UV) radiation.

Devices of the invention utilize far UV light, or radiation, that is within the spectrum of 200 nm-280 nm wavelengths to disinfect, or sanitize, a wide variety of surfaces. These devices either do not generate wavelengths of UV light outside of this range or include filters that prevent UV radiation outside of this range from being emanated into the environment. Importantly, exposure to UV light within the spectrum of 200 nm-230 nm wavelengths does not result in mutagenic or carcinogenic effects to human or animal cells. Thus, devices of the invention that only radiate UV light within the spectrum of 200 nm-230 nm wavelengths into an environment do not require shielding to be used safely.

The invention provides devices that comprise at least one light source that generates photons of at least one wavelength within the range of 200 nm to 280 nm, wherein the photons are directed at a surface such that exposing the surface to the photons achieves at least a ninety percent kill of microorganisms on the surface. Preferred wavelength ranges are between 200 nm to 230 nm or between 250 nm and 280 nm. More preferably, wavelength ranges are between 205 nm and 210 nm or between 220 nm and 225 nm. Most preferably, a light source generates photons that are predominantly at a wavelength of 207 nm or a wavelength of 222 nm.

Devices of the invention include a housing for the one or more light sources. Preferably, the housing includes a means of access, such as a door, to the interior of the housing and a means of operating the invention. The housing can be box-like or bag-like in shape. Preferably, the housing is box-like. The housing may be partially or fully transparent or partially or fully impervious to the photons that are emitted from a light source.

One or more interior surface of the housing may be wholly or partially reflective. Alternatively or in addition, one or more reflectors may be attached or applied to the interior surface of the housing. For example, aluminum or barium sulfate reflectors or coatings can be applied to the interior of the housing or to an attachable piece to cause the interior or attachable piece to become reflective. Those of skill in the art will recognize that mirrors or other reflective materials can be incorporated into the devices of the invention.

Some embodiments include a planar support that is capable of supporting a material that is to be disinfected or exposed to far UV radiation. The planar support is located within the housing. Preferably, the planar support can be accessed through a door or other means of access in the housing. The planar support can be movable or fixed. In some configurations, two or more planar support can be present in a device.

A planar support can be supported by at least one supporting member, wherein the supporting member elevates the planar support above the bottom interior surface of the housing. Suitable supporting members include at least one roller, ring, vertical support, and combination thereof such that during operation the planar support is movable. Those of skill in the art will appreciate that other types of supporting members are known in the art. The type of support member that is selected will depend, in part, upon the size and configuration of the device, as well as, the type of material(s) that are to be disinfected.

In some embodiments a planar support is elevated within the housing such that during operation photons generated by a light source are directed at a surface (e.g. a top or first side) of the planar support, or at a material placed on the planar support, and photons also are directed at one or more reflectors such that the photons are reflected towards another surface (e.g. a bottom or second side) of the planar support or the material. Preferably, an elevated planar support is constructed of fused quart, fused silica, or transparent material such that photons can pass through the planar support. Alternatively, the planar support may comprise another substance through which photons can pass so that they reach at least one surface of any material placed on the planar support. Those of skill in the art will appreciate that the light source can be located above, below, or lateral to the planar support or to a material placed on the planar support.

In some embodiments, devices of the invention comprise at least one light source that generates photons of at least one single line wavelength within the range of 200 nm to 230 nm, wherein the photons are directed at a surface such that exposing the surface to the photons achieves at least a ninety percent kill of microorganisms on the surface; a movable planar support; and a housing for the one or more light sources and the planar support. The housing includes a door to access the planar support and a means of operating the device. Such devices may include one or more reflectors. Preferably, in devices of the invention that do not include one or more reflectors, two or more light sources are present.

In certain embodiments, one or more secondary light sources can be present and situated within the device so that photons emitted from the secondary light source are directed or reflected onto one more top, side, or bottom surfaces of the planar support. Embodiments of the invention that include a secondary light source may include a planar surface that is not elevated. Rather, the planar surface may sit atop a secondary light source. Such embodiments may include one or more reflectors or no reflectors.

In some configurations, devices of the invention can include at least one second planar support. It is envisioned that these devices can be operated with either one or both planar supports in place at the same time. For example in configurations that include a reflector, during operation photons emitted by a light source are directed at upper surfaces of both a first planar support and a second planar support, and the photons are reflected towards bottom surfaces of both the first planar support and second planar support. Alternatively, a first light source may be placed above a first planar support, and secondary light sources may be placed below the second planar support and along one or more sides of the respective planar supports such that photons emitted from all light sources are directed at and through each rplanar support. One or more reflectors may be included in such embodiments.

In certain embodiments of the invention the housing may be collapsible, or partially collapsible, and either box-like or bag-like in shape. Such embodiments are envisioned to be suitable for portable or temporary uses such as camping, hiking, emergency shelters, military use and the like.

In another embodiment, portable devices may be handheld such as a wand, attached to fixtures or simply illuminated overhead or at other angles to maximize exposure.

In other embodiments, the invention provides a system for removing microorganisms from a surface comprising exposing a surface to one or more light sources that generates photons of at least one wavelength within the range of 200 nm to 280 nm, wherein the photons are directed at the surface such that at least a ninety percent kill of microorganisms on the surface are killed.

The invention also provides a method of disinfecting comprising exposing a surface to one or more light sources that generates photons of at least one wavelength within the range of 200 nm to 280 nm such that at least a ninety percent kill of microorganisms on the surface are killed.

Other objects, features and advantages of the present invention will become apparent from the following detailed description and claims. It should be understood, however, that any specific examples are given by way of illustration, and various changes and modifications that are within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is a diagram of one embodiment of the invention.

FIG. 2 is a diagram of second embodiment of the invention in a box-like configuration. Light sources are represented by short solid bars along the sides, top, and bottom of the housing, as well as, along the planar supports.

FIG. 3 is a another configuration of the invention. Light sources are represented by short solid bars that are located bars along the sides, top, and bottom of the housing, as well as, being placed within items to be disinfected.

FIG. 4 is a diagram of a handheld wand with the light source (solid bar) within the wand.

FIG. 5 is one example of an overhead lighting fixture that incorporates the invention.

FIG. 6 is an example of a configuration for a waterless sink or hand dryer.

FIG. 7 is an exemplary configuration for an enclosed box for a phone or other electronics.

FIG. 8 is a lateral view of an air or water disinfection system.

FIG. 9 is an end view of the configuration of an air or water disinfection system.

FIG. 10 is a vacuum cleaner or robotic cleaner that includes at least one light source (solid short bar) in its interior.

FIG. 11 is a diagram of exemplary lighting on an extendable mechanical arm.

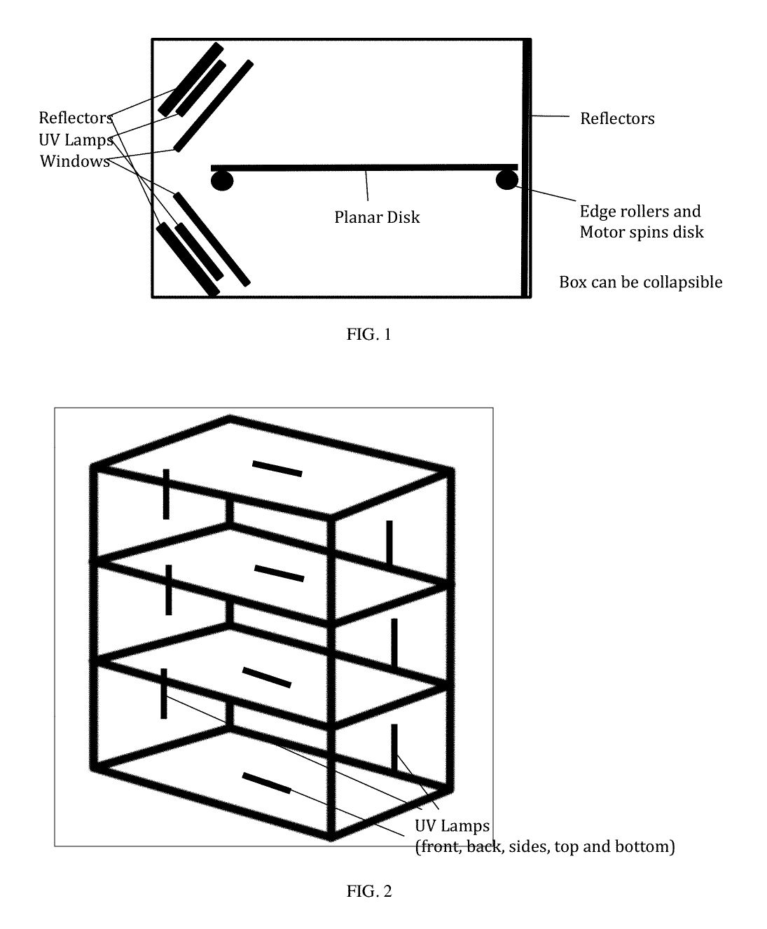

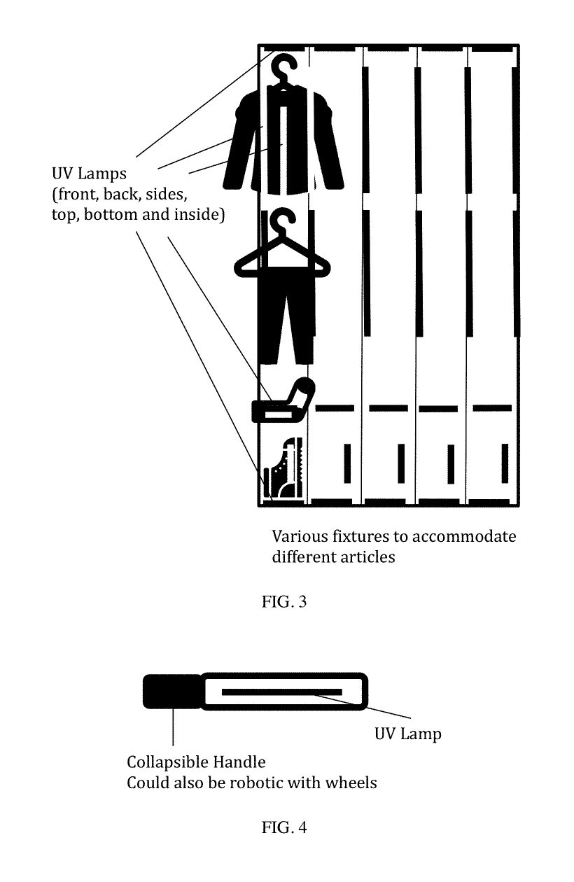

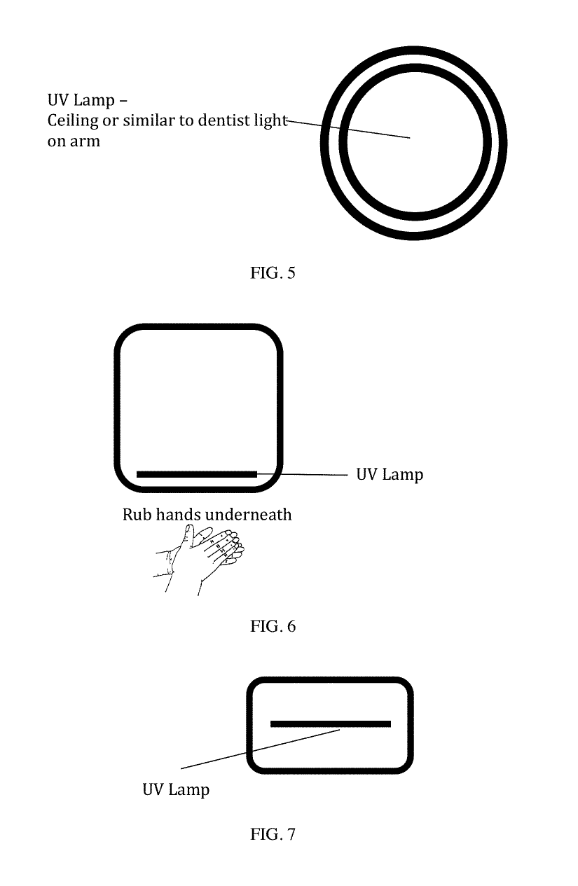

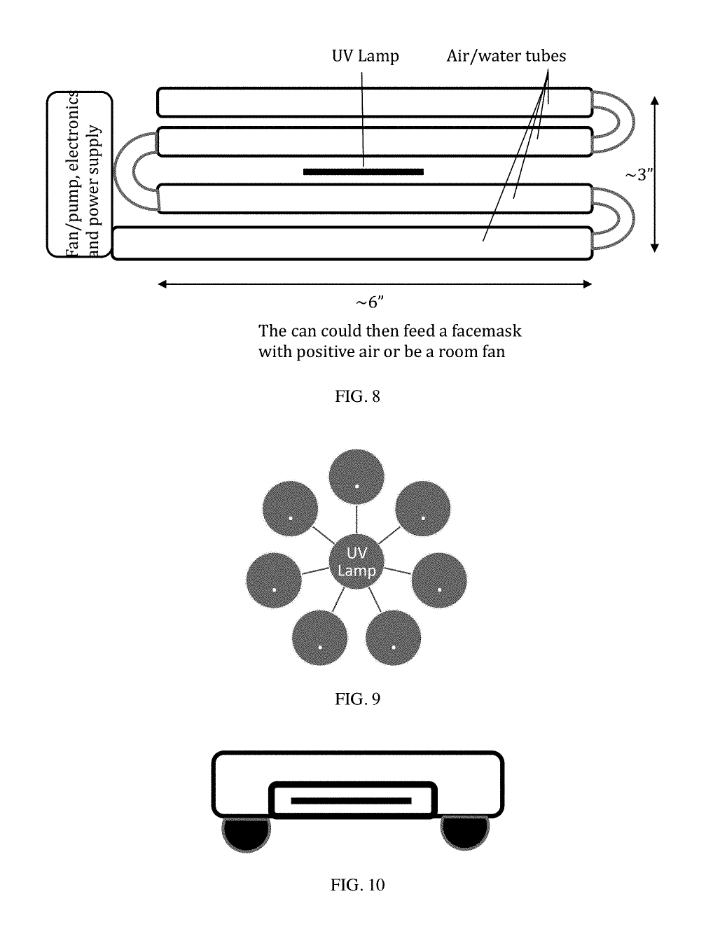

### DETAILED DESCRIPTION

The invention provides a variety of devices that emit far UV radiation that is directed towards one or more surfaces that it is desirable to disinfect from microorganisms or allergens.

The one or more light sources can be a krypton bromide lamp, krypton chloride lamp, an excimer lamp, a low-pressure, mercury-arc germicidal lamp, a dual annulus lamp, an light emitting diode (LED), or a combination thereof. In certain embodiments, plasma or xenon systems that emit UV radiation within the range of range of 200 nm to 280 nm wavelengths, or 200 nm to 230 nm wavelengths, also may be included in devices of the invention.

When a material that is placed on top of the planar support and a device of the invention is operated so that the material is exposed to the UV photons that are generated, it expected that at least 90%, 92%, 95%, 96%, 97%, 98%, 99%, 99.9%, 99.99%, 99.999%, 99.9999% or even 99.99999% or more of microorganisms on the exposed surfaces of the material will be killed. Those of skill in the art will recognize that the percentage of kill and the amount of time that it takes to achieve a desired mortality is, in part, dependent upon the distance between the material and light source(s). Thus, the skilled artisan will understand that both the distance from the light source and time of exposure can be adjusted to achieve a desired result. Similarly, the skilled artisan will understand that including multiple light sources in a device or emitting more than one wavelength of photons during operation will also effect the percentage of microorganisms killed and the amount of time required to achieve a desired mortality.

In certain embodiments, the planar support rotates in a horizontal plane during operation in a manner that is similar to a turntable. Alternatively, the planar support may wobble, or vibrate, so that material placed on the planar support can tumble or flip during operation. In embodiments that include the option for the planar support to wobble or vibrate, the planar support is configured so that material placed on the support does not fall off the support during operation.

Preferably, light sources used in the invention only emit UV radiation within the range of range of 200 nm to 230 nm wavelengths. If a light source(s) generates UV radiation greater than 230 nm wavelengths, then one or more filters can be included such that over 99% of UV radiation greater than 230 nm wavelengths is absorbed by the filter(s) and is not emitted into the surrounding environment. For example, a coating on a reflector, planar surface, interior of the housing, or a combination thereof may be used to absorb harmful wavelengths. Preferably, a filter(s) is directly attached or adjacent to the light source so that only UV radiation within the range of range of 200 nm to 230 nm wavelengths is emitted into the environment from the light source.

It is envisioned that devices of the invention can be used for a wide variety of purposes in multiple industries. For example, they may be incorporated into safety protocols associated with the use or manufacture of electronics, computers, telephones, mobile devices, and the like. Devices of the invention may be used to disinfect appliances, countertops, laundry, textiles, floors, rugs, dishes, utensils and their holders, vacuum cleaners (e.g. Roomba®), and food storage containers.

Certain embodiments of the invention may be used to disinfect or sanitize hands without the aid of soap, water, or towels. For example, such devices can be used in the place of sinks in either public or household environments, particularly in locations where water may not be easily or inexpensively accessible. Advantageously, less waste, such as used paper towels, would be generated in such environments, which could assist in reducing the transmission of disease-causing organisms.

Alternatively, devices of the invention may be useful in food processing or preparation. For example, spoilage may be reduced during preparation or packaging, or alternatively, use at the retail level may delay spoilage and increase the length of shelf life. Because devices can be configured so that the amount of time required to disinfect surfaces is often less than minute, consumers may wish to use such devices before leaving a grocery store on perishable items. In other configurations, devices of the invention can be used at salad or food bars to reduce the potential of spreading disease or infection.

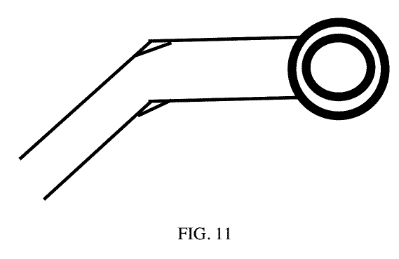

Portable devices may be used to disinfect breathable air. For example, respirators or powered air masks may include embodiments of the present invention. Alternatively, head lamps, overhead lighting, or auxiliary lighting (FIG. 11) may include embodiments of the present invention. Preferably, such embodiments do not generate any UV spectrum wavelengths that are known to be harmful to human or animal cells such that a filter(s) is necessary for safe use. Breathing apparati can also be fully contained and utilize UVC wavelengths.

It is envisioned that either portable or permanently affixed devices of the invention may be useful for the treatment of skin conditions that are associated with microorganisms such as acne, psoriasis, other skin diseases, biopsies, or eye-related treatments.

Devices of the invention provide an improved method for producing a dry, chemical free, disinfection of food stuffs. Furthermore, it can be used to disinfect cutting and working surfaces for meat and poultry, as well as, the equipment used to prepare or transport meat and poultry and other food products.

The far UV radiation can be configured to selectively affect or destroy at least one microorganism on a surface, while substantially avoiding harm to human or animal cells. The light source of the far UV radiation can include a krypton-bromine lamp or a krypton-chlorine lamp excilamp. Optionally, either a narrow band of wavelengths of far UV radiation can be emitted from a device or a broader distribution of wavelengths of far UV radiation can be emitted. For example, wavelengths can be between 200 nm and 280 nm. Preferred wavelengths are between 200 nm and 230 nm. More preferably, wavelengths are between 205 nm and 210 nm or between 220 nm and 225 nm. Most preferably, wavelengths are predominantly at 207 nm or 222 nm.

Certain exemplary features can be included (e.g., spectrum filtering elements such as multilayer dielectric filters or chemical filters) to remove unwanted wavelengths, or those wavelengths that are outside of the preferable range of wavelengths. For example, absorption and/or reflective elements can be provided between the lamp and the irradiated surface to filter unwanted wavelengths, such as, e.g., a band-pass filter, a long-wavelength blocking filter.

Distance also plays a factor into the efficacy of UV light as a disinfectant. Following the inverse square law, the strength of UV radiation will decrease as it gets further away from its source. For example, far UV light will have one quarter of its power when it is twice the distance from its source that it had at the original reference point. This relationship limits how far a single source of UV light will be effective before it is too weak to provide adequate disinfection. The UV dose is the product of UV intensity [I] (expressed as energy per unit surface area) and exposure time [T]. Therefore: DOSE=I×T. This dose, sometimes referred to as fluence, is commonly expressed as millijoule per square centimeter (mJ/cm²). The units “J/m²” are used in most parts of the world except for North America, where “mJ/cm²” are used. Thus, for any given configuration of a device of the present invention, it is possible to calculate the UV light intensity that will be achieved.

Further, the reduction of many different types of microorganisms is classified using a logarithmic scale and is known. For example, a single log reduction is a 90% reduction of organisms; a two log reduction is a 99% reduction of organisms; a three log reduction is a 99.9% reduction of organisms; and a seven log reduction is up to a 99.99999% reduction of organisms. Thus, for any particular configuration of a device of the present invention it is possible to determine the amount of time of exposure that is required to achieve the desired amount of kill for various microorganisms based on the amount UV light intensity that can be achieved at the point of contact of the surface that is to be treated.

Unless defined otherwise, all technical and scientific terms used herein have the same meaning as is commonly understood by one of skill in the art to which this invention belongs at the time of filing. The meaning and scope of terms should be clear; however, in the event of any latent ambiguity, definitions provided herein take precedent over any dictionary or extrinsic definition. Further, unless otherwise required by context, singular terms shall include pluralities and plural terms shall include the singular. Herein, the use of “or” means “and/or” unless stated otherwise. Furthermore, the use of the term “including”, as well as other forms such as “includes” and “included” is not limiting. All patents and publications referred to herein are incorporated by reference herein.

It is to be understood that the terminology used herein is for the purpose of describing particular embodiments of the invention only, and is not intended to be limiting. It must be noted that, as used in this specification and the appended claims, the singular forms “a”, “an” and “the” include plural referents unless the content clearly dictates otherwise.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the following claims.

What is claimed is:

1. A device comprising
a) one or more light sources that generates photons of at least one wavelength within the range of 200 nm to 280 nm, wherein the photons are directed at a surface such that exposing the surface to the photons achieves at least a ninety percent kill of microorganisms on the surface; and
b) a housing for the one or more light sources, wherein the housing includes a means to access the interior of the housing and a means of operating the one or more light sources.

2. The device of claim 1 further comprising a planar support of fused quartz, fused silica or other substantially transparent material.

3. The device of claim 2, wherein the planar support is elevated within the housing such that during operation photons generated by a light source are directed at an upper surface of the planar support.

4. The device of claim 2, wherein the planar support is fixed in position.

5. The device of claim 2, wherein the planar support is movable.

6. The device of claim 1 further comprising one or more reflectors positioned such that the photons are reflected towards a top, side, or bottom surface of a planar support.

7. The device of claim 1 further comprising one or more reflectors positioned such that the photons are reflected towards a top, side, or bottom surface of a material positioned within the housing.

8. The device of claim 1, wherein the one or more light sources is a krypton bromide lamp, krypton chloride lamp, an excimer lamp, light emitting diode (LED), or a low-pressure, mercury-arc germicidal lamp.

9. The device of claim 2, wherein exposing a material placed on top of the planar support to the photons achieves at least a ninety five percent kill of microorganisms on exposed surfaces of the material.

10. The device of claim 1, wherein at least a ninety nine percent of the microorganisms are killed.

11. The device of claim 2 further comprising at least one supporting member, wherein the supporting member elevates the planar support above the bottom interior surface of the housing.

12. The device of claim 5, wherein the planar support rotates in a horizontal plane during operation.

13. The device of claim 12 further comprising at least one roller that rotates the planar support during operation.

14. The device of claim 2 further comprising a second planar support such that during operation photons are directed at upper surfaces of both a first planar support and a second planar support, and photons are reflected towards at least one side or bottom surface of both the first planar support and the second planar support.

15. The device of claim 1, wherein the photons generated are of at least one wavelength within the range of 200 nm to 230 nm.

16. A system for removing microorganisms from a surface comprising exposing a surface to one or more light sources that generates photons of at least one wavelength within the range of 200 nm to 280 nm, wherein the photons are directed at the surface such that at least a ninety percent kill of microorganisms on the surface are killed.

17. A method of disinfection comprising exposing a surface to one or more light sources that generates photons of at least one wavelength within the range of 200 nm to 280 nm such that at least a ninety percent kill of microorganisms on the surface are killed.

18. The method of claim 17, wherein the one or more light sources is enclosed within a housing, wherein the housing includes a means to access the interior of the housing and a means of operating the one or more light sources.
